(19) United States
(12) Patent Application Publication
(10) Pub. No.: US 2026/0295208 A1
(43) Pub. Date: Oct. 1, 2026

(54) STEERABLE SHEATH ASSEMBLY AND METHODS OF MAKING A STEERABLE SHEATH

(71) Applicant: BOSTON SCIENTIFIC SCIMED, INC., MAPLE GROVE, MN (US)

(72) Inventors: Nicholas Lee Tassoni, Andover, MN (US); Andrew Denzer, Plymouth, MN (US); Andrea Goering, Blaine, MN (US)

(73) Assignee: BOSTON SCIENTIFIC SCIMED, INC., MAPLE GROVE, MN (US)

(21) Appl. No.: 19/578,306

(22) Filed: Mar. 25, 2026

Related U.S. Application Data
(60) Provisional application No. 63/777,156, filed on Mar. 25, 2025.

(51) Int. Cl. A61M 25/00; A61M 25/01

(52) U.S. Cl. CPC A61M 25/0009; A61M 25/0045; A61M 25/005; A61M 25/0138

(57) ABSTRACT

A method of making a steerable sheath assembly includes disposing at least one tubular member alongside an inner sleeve, each tubular member of the at least one tubular member having a core wire disposed therein; disposing a reinforcing braid over the inner sleeve and the at least one tubular member; disposing an outer sleeve over the reinforcing braid; proximally withdrawing the core wire only partially from its respective tubular member; and overmolding a proximal hub structure onto the outer sleeve or onto the outer sleeve and a proximal portion of each tubular member. The core wire of each tubular member or the proximal portion of each tubular member extends through a distal portion of the proximal hub structure to an exterior of the proximal hub structure at a location distal of a proximal end of the proximal hub structure.

### CROSS-REFERENCE TO RELATED APPLICATIONS

This application claims the benefit of priority of U.S. Provisional Application No. 63/777,156 filed Mar. 25, 2025, the entire disclosure of which is hereby incorporated by reference.

### TECHNICAL FIELD

The disclosure relates generally to medical devices and more particularly to steerable sheath assemblies and/or methods of making steerable sheath assemblies.

### BACKGROUND

A wide variety of intracorporeal medical devices have been developed for medical use, and more specifically surgical and/or intravascular use. Some of these devices include guidewires, catheters, medical device delivery systems (e.g., for stents, grafts, replacement valves, etc.), and the like. These devices are manufactured by any one of a variety of different manufacturing methods and may be used according to any one of a variety of methods. There is an ongoing need to provide alternative medical devices as well as alternative methods for manufacturing and/or using medical devices.

### SUMMARY

In one example, a method of making a steerable sheath assembly may comprise positioning an inner tubular sleeve on a mandrel; disposing at least one tubular member alongside the inner tubular sleeve, each tubular member of the at least one tubular member having a core wire disposed therein; disposing a reinforcing braid over the inner tubular sleeve and the at least one tubular member; disposing an outer tubular sleeve over the reinforcing braid; proximally withdrawing the core wire from each tubular member of the at least one tubular member only partially from its respective tubular member; and overmolding a proximal hub structure onto a proximal end of the outer tubular sleeve. The core wire of each tubular member of the at least one tubular member may extend through a distal portion of the proximal hub structure to an exterior of the proximal hub structure at a location distal of a proximal end of the proximal hub structure.

In addition, or alternatively, to any example disclosed herein, the method may comprise removing the core wire of each tubular member of the at least one tubular member from the distal portion of the proximal hub structure thereby defining a separate lumen extending from each tubular member of the at least one tubular member through the distal portion of the proximal hub structure to the exterior of the proximal hub structure.

In addition, or alternatively, to any example disclosed herein, the method may comprise positioning a steering wire within each tubular member of the at least one tubular member such that each steering wire extends distal from its respective tubular member of the at least one tubular member, through its respective tubular member of the at least one tubular member, and exits to the exterior of the proximal hub structure through its respective separate lumen; and attaching a steering ring to the outer tubular sleeve proximate a distal end of the reinforcing braid. Each steering wire may be fixedly attached to the steering ring.

In addition, or alternatively, to any example disclosed herein, each separate lumen extending through the distal portion of the proximal hub structure, at a proximal end thereof, forms an angle relative to a central longitudinal axis of the proximal hub structure of between about 0 degrees and about 35 degrees.

In addition, or alternatively, to any example disclosed herein, each separate lumen extending through the distal portion of the proximal hub structure curves within the distal portion of the proximal hub structure along a radius of between about 0.075 inches (1.91 mm) and about 0.500 inches (12.7 mm).

In addition, or alternatively, to any example disclosed herein, each tubular member of the at least one tubular member does not extend proximal of a proximal end of the outer tubular sleeve.

In addition, or alternatively, to any example disclosed herein, the method may comprise disposing a helical coil around the inner tubular sleeve prior to disposing the at least one tubular member alongside the inner tubular sleeve such that the helical coil is disposed between the at least one tubular member and the inner tubular sleeve.

In addition, or alternatively, to any example disclosed herein, and in a second example, a steerable sheath assembly may comprise an inner tubular sleeve, at least one tubular member positioned alongside the inner tubular sleeve, a reinforcing braid disposed over the inner tubular sleeve and the at least one tubular member, an outer tubular sleeve disposed over the reinforcing braid, and a proximal hub structure overmolded onto the outer tubular sleeve. The proximal hub structure may comprise a separate lumen extending from each tubular member of the at least one tubular member through a distal portion of the proximal hub structure to an exterior of the proximal hub structure at a location distal of a proximal end of the proximal hub structure.

In addition, or alternatively, to any example disclosed herein, the steerable sheath assembly may comprise a helical coil between the inner tubular sleeve and the at least one tubular member.

In addition, or alternatively, to any example disclosed herein, the steerable sheath assembly may comprise a steering wire disposed within each tubular member of the at least one tubular member such that each steering wire extends from the exterior of the proximal hub structure to distal of the at least one tubular member, and a steering ring disposed proximate a distal end of the reinforcing braid. Each steering wire may be fixedly attached to the steering ring.

In addition, or alternatively, to any example disclosed herein, each separate lumen extending through the distal portion of the proximal hub structure, at a proximal end thereof, forms an angle relative to a central longitudinal axis of the proximal hub structure of between about 0 degrees and about 35 degrees.

In addition, or alternatively, to any example disclosed herein, each separate lumen extending through the distal portion of the proximal hub structure curves within the distal portion of the proximal hub structure along a radius of between about 0.075 inches (1.91 mm) and about 0.500 inches (12.7 mm).

In addition, or alternatively, to any example disclosed herein, the proximal hub structure comprises a proximal portion including a fluid port spaced apart from the distal portion, and a tubular medial portion extending from the proximal portion of the proximal hub structure to the distal portion of the proximal hub structure.

In addition, or alternatively, to any example disclosed herein, and in a third example, a method of making a steerable sheath assembly may comprise positioning an inner tubular sleeve on a mandrel; disposing at least one tubular member alongside the inner tubular sleeve, each tubular member of the at least one tubular member having a core wire disposed therein; disposing a reinforcing braid over the inner tubular sleeve and the at least one tubular member; disposing an outer tubular sleeve over the reinforcing braid; and overmolding a proximal hub structure onto a proximal end of the outer tubular sleeve and a proximal portion of each tubular member of the at least one tubular member. The proximal portion of each tubular member of the at least one tubular member may extend through a distal portion of the proximal hub structure to an exterior of the proximal hub structure at a location distal of a proximal end of the proximal hub structure.

In addition, or alternatively, to any example disclosed herein, the method may comprise, before disposing the reinforcing braid over the inner tubular sleeve and the at least one tubular member, positioning a steering wire within each tubular member of the at least one tubular member such that each steering wire extends from proximal of its respective tubular member to distal of its respective tubular member; and positioning a steering ring over inner tubular sleeve distal of the at least one tubular member. Each steering wire may be fixedly attached to the steering ring.

In addition, or alternatively, to any example disclosed herein, the method may comprise positioning a steering wire within each tubular member of the at least one tubular member such that each steering wire extends from proximal of its respective tubular member to distal of its respective tubular member; and attaching a steering ring to the outer tubular sleeve proximate a distal end of the reinforcing braid. Each steering wire may be fixedly attached to the steering ring.

In addition, or alternatively, to any example disclosed herein, each tubular member of the at least one tubular member comprises a core wire disposed therein when disposing the at least one tubular member alongside the inner tubular sleeve.

In addition, or alternatively, to any example disclosed herein, the method may comprise, after overmolding the proximal hub structure and before positioning the steering wire within each tubular member of the at least one tubular member, removing the core wire from each tubular member of the at least one tubular member.

In addition, or alternatively, to any example disclosed herein, the proximal portion of each tubular member of the at least one tubular member at a proximal end thereof forms an angle relative to a central longitudinal axis of the proximal hub structure of between about 0 degrees and about 35 degrees.

In addition, or alternatively, to any example disclosed herein, the proximal portion of each tubular member of the at least one tubular member curves within the distal portion of the proximal hub structure along a radius of between about 0.075 inches (1.91 mm) and about 0.500 inches (12.7 mm).

The above summary of some embodiments, aspects, and/or examples is not intended to describe each embodiment or every implementation of the present disclosure. The figures and the detailed description more particularly exemplify aspects of these embodiments.

### BRIEF DESCRIPTION OF THE DRAWINGS

The disclosure may be more completely understood in consideration of the following detailed description in connection with the accompanying drawings, in which:

FIGS. 1-11 schematically illustrate selected aspects of a steerable sheath assembly and selected aspects of methods of making the steerable sheath assembly;

FIGS. 12-14 schematically illustrate selected aspects of a steerable sheath assembly and selected aspects of methods of making the steerable sheath assembly; and

FIGS. 15-16 schematically illustrate selected aspects of a steerable sheath assembly and selected aspects of methods of making the steerable sheath assembly.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently – such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Disclosed herein are selected aspects of steerable sheath assemblies and methods of making steerable sheath assemblies. In some embodiments, the steerable sheath assemblies may be any one of a variety of steerable sheaths and/or catheters, such as an intravascular catheter. Examples of intravascular catheters may include, but are not limited to, balloon catheters, atherectomy catheters, device delivery catheters, drug delivery catheters, diagnostic catheters, and guide catheters. In some embodiments, the steerable sheath assemblies may take the form of other suitable guiding, diagnosing, or treating devices (including endoscopic instruments, laparoscopic instruments, etc., and the like) and it may be suitable for use at various locations and/or body lumens within a patient. Existing devices and/or methods may have certain advantages and/or disadvantages. There is an ongoing need for alternative steerable sheath assemblies and methods of making steerable sheath assemblies.

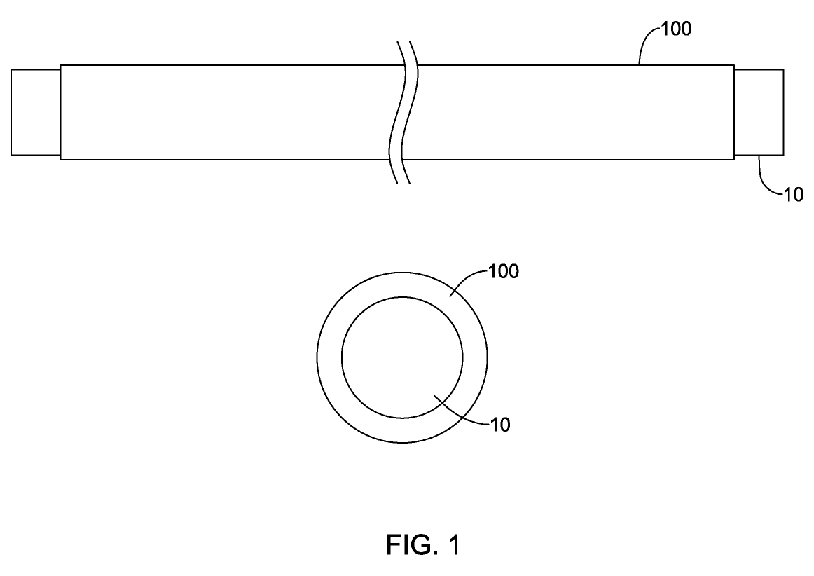

FIGS. 1-11 illustrate selected aspects of a steerable sheath assembly and a method of making the steerable sheath assembly. To improve clarity, some elements of the steerable sheath assembly are not shown in every figure or are shown schematically. In some embodiments, a method of making a steerable sheath assembly may comprise positioning an inner tubular sleeve 100 on a mandrel or a core 10, as seen in FIG. 1. In some embodiments, the inner tubular sleeve 100 may have a proximal end and a distal end (e.g., the inner tubular sleeve 100 may be formed as a discrete element). In some embodiments, the inner tubular sleeve 100 may be formed as a continuous element and after construction of the steerable sheath assembly is completed, the steerable sheath assembly may be cut to length as desired. Other configurations are also contemplated. In some embodiments, the inner tubular sleeve 100 may comprise an outer surface, an inner surface, and a central lumen defined by the inner surface. In some embodiments, the method of making the steerable sheath assembly may comprise positioning the inner surface of the inner tubular sleeve 100 over, along, and/or against an outer surface of the mandrel or the core 10. In some embodiments, the mandrel or the core 10 may be formed from a polymeric material such as acetal (e.g., polyoxymethylene (POM)). In some embodiments, the mandrel or the core 10 may be formed from a metallic material such as stainless steel. Other configurations and/or materials are also contemplated. Some suitable but non-limiting examples of materials that may be used for the mandrel or the core 10, including polymeric materials, metallic materials, composites, and the like, are described below.

In some embodiments, the inner tubular sleeve 100 may be formed from a polymeric material. In some embodiments, the inner tubular sleeve 100 may be formed from two or more layers of polymeric material, wherein the two or more layers of polymeric material may each be different and/or discrete polymeric materials. In some embodiments, the two or more layers of polymeric material may be coextruded. In some embodiments, the two or more layers of polymeric material may be adhesively and/or molecularly bonded together. Other configurations are also contemplated. In one example, the inner tubular sleeve 100 may comprise a polytetrafluoroethylene (PTFE) liner or layer with a polyether block amide (e.g., Pebax®) layer disposed thereon and/or radially outward of the PTFE liner or layer. Other configurations and/or polymeric materials are also contemplated, and some non-limiting examples are described below.

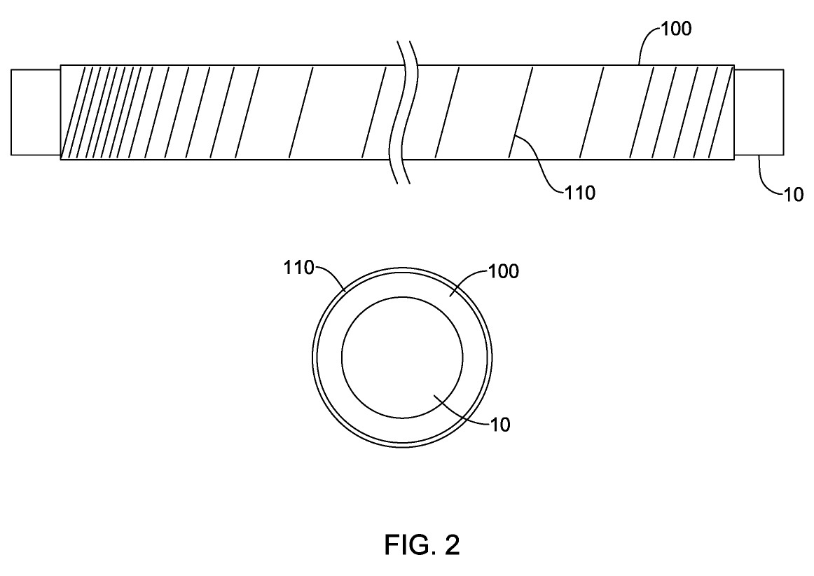

In some embodiments, the method of making the steerable sheath assembly may optionally comprise disposing a helical coil 110 around the inner tubular sleeve 100, as seen in FIG. 2. In some embodiments, the helical coil 110 may be formed with a variable pitch (e.g., a variable distance between adjacent windings of the helical coil 110) along the length of the inner tubular sleeve 100. In some alternative configurations, the helical coil 110 may be formed with a constant pitch (e.g., a uniform, constant, and/or non-variable distance between adjacent windings of the helical coil 110) along the length of the inner tubular sleeve 100. In some embodiments, the helical coil 110 may be configured to impart desired and/or particular characteristics to the steerable sheath assembly. In some embodiments, the helical coil 110 may have a closed pitch and/or a tighter pitch proximate a proximal end of the steerable sheath assembly. In some embodiments, the helical coil 110 may have an open pitch and/or a wider pitch along a medial portion of the steerable sheath assembly. In some embodiments, the medial portion of the steerable sheath assembly may be configured for and/or may be intended to facilitate bending and/or deflection of the steerable sheath assembly. In some embodiments, the helical coil 110 may have a medium pitch (e.g., a pitch between the closed and/or tighter pitch and the open and/or wider pitch) proximate a distal end of the steerable sheath assembly. Other configurations are also contemplated. For example, the helical coil 110 may have a pitch that increases or decreases gradually in one direction along the length of the steerable sheath assembly. In some embodiments, the helical coil 110 may be formed from a metallic material such as stainless steel. Other configurations and/or materials are also contemplated. Some suitable but non-limiting examples of materials that may be used for the helical coil 110, including polymeric materials, metallic materials, composites, and the like, are described below.

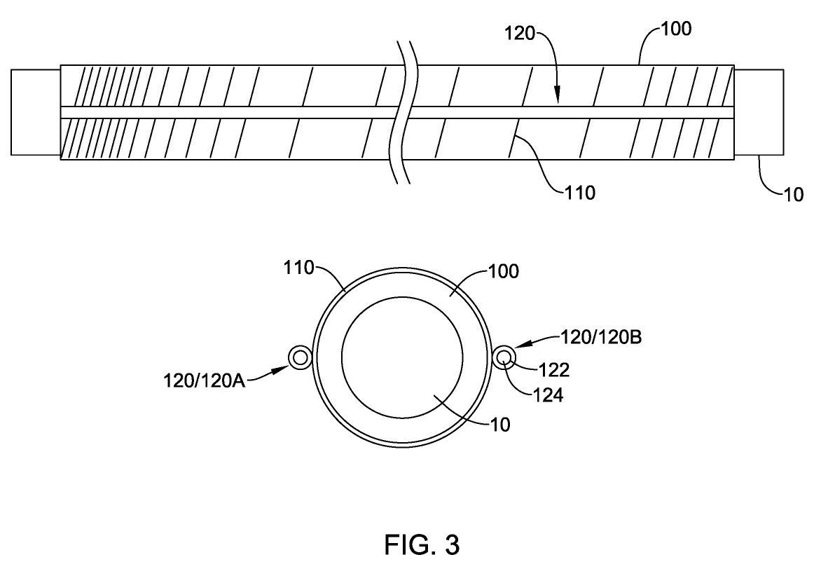

In some embodiments, the method of making the steerable sheath assembly may comprise disposing at least one tubular member 120 alongside the inner tubular sleeve 100 and/or the helical coil 110, as seen in FIG. 3. In some embodiments, the method of making the steerable sheath assembly may comprise disposing the helical coil 110 around the inner tubular sleeve 100 prior to disposing the at least one tubular member 120 alongside the inner tubular sleeve 100 such that the helical coil 110 is disposed between the at least one tubular member 120 and the inner tubular sleeve 100.

In some embodiments, the at least one tubular member 120 may comprise two tubular members. In some embodiments, the at least one tubular member 120 may comprise four tubular members. Other configurations are also contemplated. In some embodiments, the number of tubular members in the steerable sheath assembly may be determined by how many different directions of movement and/or steering are desired.

In some embodiments, where the at least one tubular member 120 comprises two tubular members, the two tubular members (e.g., a first tubular member 120A and a second tubular member 120B) may be disposed on opposite sides of the inner tubular sleeve 100, as seen in FIG. 3. In some embodiments, where the at least one tubular member 120 comprises four tubular members, the four tubular members may be disposed in opposing pairs arrayed and/or equidistantly spaced around the circumference of the inner tubular sleeve 100. Other configurations are also contemplated.

In some embodiments, the at least one tubular member 120 may comprise a polymeric tubular member. In one non-limiting example, the polymeric tubular member may comprise high density polyethylene (HDPE). Some suitable but non-limiting examples of polymeric materials for the polymeric tubular member are described below. In some embodiments, the at least one tubular member 120 may comprise a tube reinforcing braid disposed on and/or embedded within the polymeric tubular member. In some embodiments, the tube reinforcing braid may be formed from a metallic material. In one non-limiting example, the tube reinforcing braid may comprise stainless steel. Other configurations and/or materials are also contemplated. Some suitable but non-limiting examples of materials that may be used for the tube reinforcing braid, including polymeric materials, metallic materials, composites, and the like, are described below.

In some embodiments, each tubular member of the at least one tubular member 120 may define a steering wire lumen 122 extending therein and/or therethrough. In some embodiments, the at least one tubular member 120 may be formed on and/or may comprise a core wire 124 extending within the steering wire lumen 122. In some embodiments, the core wire 124 may be formed from a metallic material. In one non-limiting example, the core wire 124 may be formed from stainless steel. In some embodiments, the core wire 124 may be formed from a material that does not bond well (or at all) to the polymeric tubular member such that the core wire 124 may be removed from the steering wire lumen 122 with little or no effort (e.g., specialized tooling and/or mechanical manipulation is not required to remove the core wire 124). Other configurations and/or materials are also contemplated. Some suitable but non-limiting examples of materials that may be used for the core wire 124, including polymeric materials, metallic materials, composites, and the like, are described below. In some embodiments, each tubular member of the at least one tubular member 120 may comprise the core wire 124 disposed therein when disposing the at least one tubular member 120 alongside the inner tubular sleeve 100 and/or the helical coil 110.

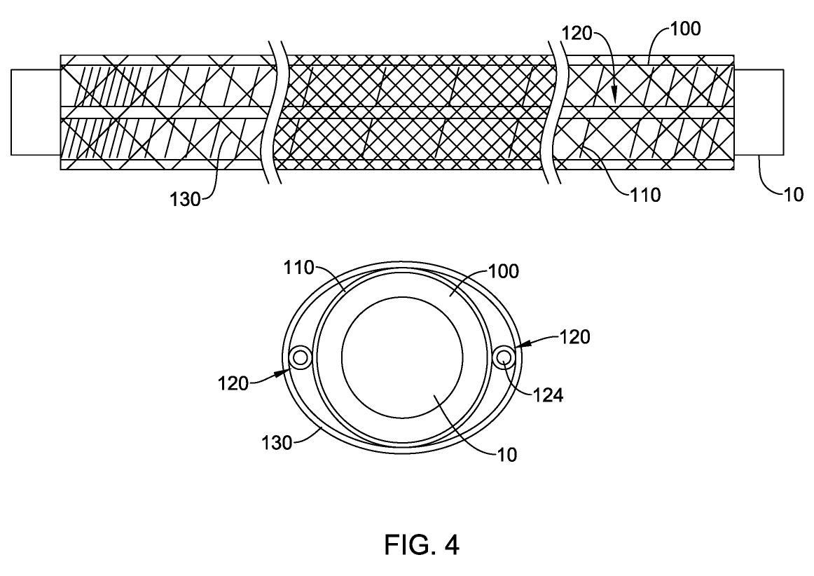

In some embodiments, the method of making a steerable sheath assembly may comprise disposing a reinforcing braid 130 over the inner tubular sleeve 100 and the at least one tubular member 120, as seen in FIG. 4. In some embodiments, the reinforcing braid 130 may be formed with a variable braid pattern (e.g., pick count; picks per inch (PPI)) along the length of the reinforcing braid 130. In some alternative configurations, the reinforcing braid 130 may be formed with a constant braid pattern along the length of the reinforcing braid 130. In some embodiments, the reinforcing braid 130 may be configured to impart desired and/or particular characteristics to the steerable sheath assembly. In some embodiments, the reinforcing braid 130 may have a closed pattern and/or a higher pick count along a medial portion of the steerable sheath assembly. In some embodiments, the reinforcing braid 130 may have an open patten and/or a lower pick count proximate a proximal end of the steerable sheath assembly. In some embodiments, the medial portion of the steerable sheath assembly may be configured for and/or may be intended to facilitate bending and/or deflection of the steerable sheath assembly. In some embodiments, the reinforcing braid 130 may have a medium pattern (e.g., an intermediate pick count between the higher pick count and the lower pick count) proximate a distal end of the steerable sheath assembly. Other configurations are also contemplated. For example, the reinforcing braid 130 may have a pick count that increases or decreases gradually in one direction along the length of the steerable sheath assembly. In some embodiments, the reinforcing braid 130 may be formed from a metallic material such as stainless steel. Other configurations and/or materials are also contemplated. Some suitable but non-limiting examples of materials that may be used for the reinforcing braid 130, including polymeric materials, metallic materials, composites, and the like, are described below.

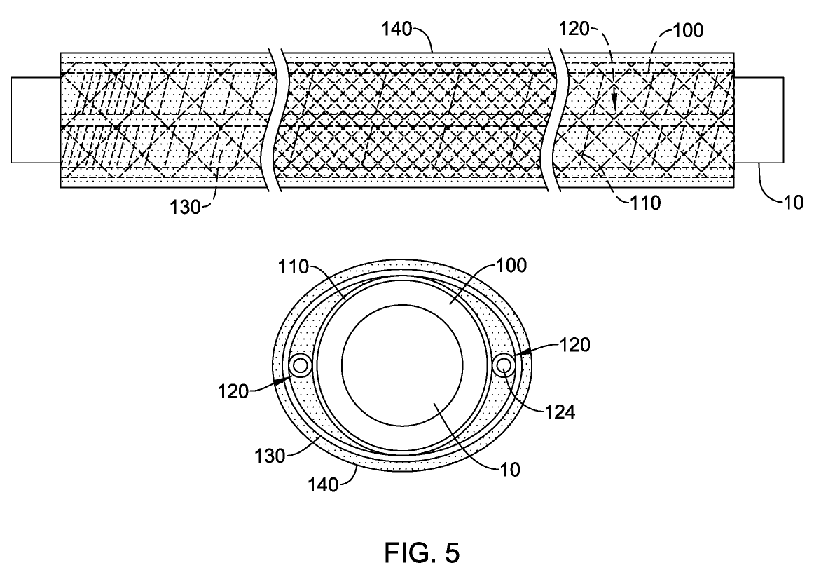

In some embodiments, the method of making the steerable sheath assembly may comprise disposing an outer tubular sleeve 140 over the reinforcing braid 130, as seen in FIG. 5. For reference, the outer tubular sleeve 140 is shown with dotted shading. In some embodiments, disposing an outer tubular sleeve 140 over the reinforcing braid 130 may comprise co-extruding and/or co-molding the outer tubular sleeve 140 over and/or onto the inner tubular sleeve 100, the helical coil 110, the at least one tubular member 120, and/or the reinforcing braid 130. In some embodiments, disposing an outer tubular sleeve 140 over the reinforcing braid 130 may comprise embedding the reinforcing braid 130 within the outer tubular sleeve 140. In some embodiments, disposing the outer tubular sleeve 140 over the reinforcing braid 130 may cause the outer tubular sleeve 140 to fill in around the inner tubular sleeve 100, the helical coil 110, the at least one tubular member 120, and/or the reinforcing braid 130.

In some embodiments, the outer tubular sleeve 140 may be formed from a polymeric material. In one example, the outer tubular sleeve 140 may comprise a polyether block amide (e.g., Pebax®). Other configurations and/or polymeric materials are also contemplated, and some non-limiting examples are described below. In some alternative embodiments, the outer tubular sleeve 140 may be formed from two or more layers of polymeric material, wherein the two or more layers of polymeric material may each be different and/or discrete polymeric materials. In some embodiments, the two or more layers of polymeric material may be coextruded. In some embodiments, the two or more layers of polymeric material may be adhesively and/or molecularly bonded together. Other configurations are also contemplated.

In some embodiments, the steerable sheath assembly and/or the outer tubular sleeve 140 may have an outer shape that is ovoid. However, this is not intended to be limiting. In some embodiments, the tooling and/or process used to dispose the outer tubular sleeve 140 over and/or onto the inner tubular sleeve 100, the helical coil 110, the at least one tubular member 120, and/or the reinforcing braid 130 may produce an outer shape of the steerable sheath assembly and/or the outer tubular sleeve 140 that is circular. Other configurations and/or shapes, including but not limited to flattened, polygonal (e.g., triangular, square, rectangular, trapezoidal, pentagonal, hexagonal, octagonal, etc.), irregular, other shapes, and/or combinations thereof, are also contemplated.

In some embodiments, each tubular member of the at least one tubular member 120 may not extend proximal of a proximal end of the outer tubular sleeve 140. In some embodiments, the steerable sheath assembly may be constructed such that all elements of the steerable sheath assembly terminate at a single longitudinal position at the proximal end of the outer tubular sleeve 140. In some embodiments, the steerable sheath assembly may be trimmed to length such that all elements of the steerable sheath assembly terminate at a single longitudinal position at the proximal end of the outer tubular sleeve 140. Other configurations are also contemplated.

In some embodiments, each tubular member of the at least one tubular member 120 may not extend distal of a distal end of the outer tubular sleeve 140. In some embodiments, the steerable sheath assembly may be constructed such that all elements of the steerable sheath assembly terminate at a single longitudinal position at the distal end of the outer tubular sleeve 140. In some embodiments, the steerable sheath assembly may be trimmed to length such that all elements of the steerable sheath assembly terminate at a single longitudinal position at the distal end of the outer tubular sleeve 140. Other configurations are also contemplated.

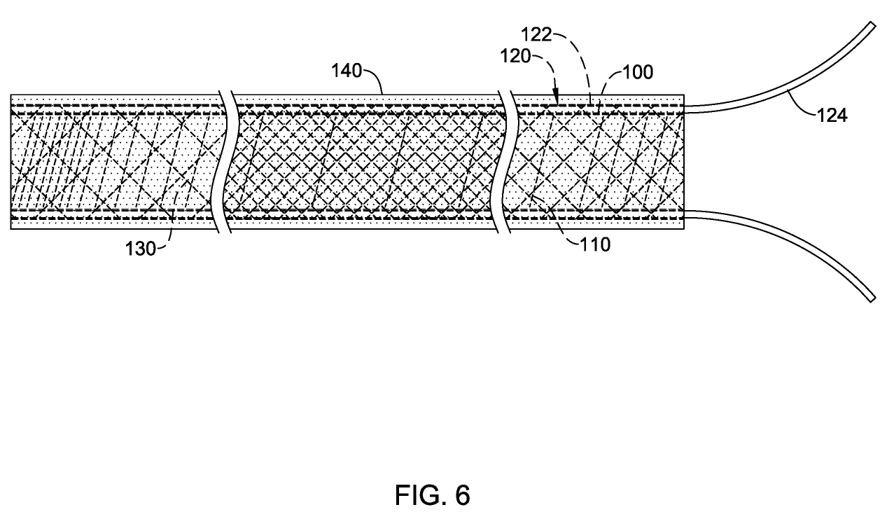

In some embodiments, the method of making the steerable sheath assembly may comprise proximally translating the core wire 124 proximally within each tubular member of the at least one tubular member 120, as seen in FIG. 6. In some embodiments, the method of making the steerable sheath assembly may comprise proximally translating only a portion of the core wire 124 proximally out of a proximal end of each tubular member of the at least one tubular member 120. In some embodiments, the method of making the steerable sheath assembly may comprise proximally translating the core wire 124 proximally within each tubular member of the at least one tubular member 120 such that a first portion (e.g., a proximal portion) of the core wire 124 is disposed proximally of the steering wire lumen 122 of each tubular member of the at least one tubular member 120 while a second portion (e.g., a distal portion) of the core wire 124 remains and/or is maintained within the steering wire lumen 122 of its respective tubular member of the at least one tubular member 120.

In some embodiments, the method of making the steerable sheath assembly may comprise proximally withdrawing a portion of the core wire 124 from each tubular member of the at least one tubular member 120. In some embodiments, the method of making the steerable sheath assembly may comprise proximally withdrawing the core wire 124 from each tubular member of the at least one tubular member 120 only partially from its respective tubular member of the at least one tubular member 120. In some embodiments, the method of making the steerable sheath assembly may comprise positioning a first portion (e.g., a proximal portion) of the core wire 124 proximally of the steering wire lumen 122 of each tubular member of the at least one tubular member 120 while maintaining a second portion (e.g., a distal portion) of the core wire 124 within the steering wire lumen 122 of its respective tubular member of the at least one tubular member 120.

In some embodiments, the method of making the steerable sheath assembly may comprise pushing a portion of the core wire 124 proximally out of each tubular member of the at least one tubular member 120. In one example, the core wire 124 may be pushed proximally by squeezing a distal end and/or a distal portion of the at least one tubular member 120. In another example, the core wire 124 may be pushed proximally by inserting a tool or a pusher member into a distal end and/or a distal portion of the at least one tubular member 120. Other configurations are also contemplated.

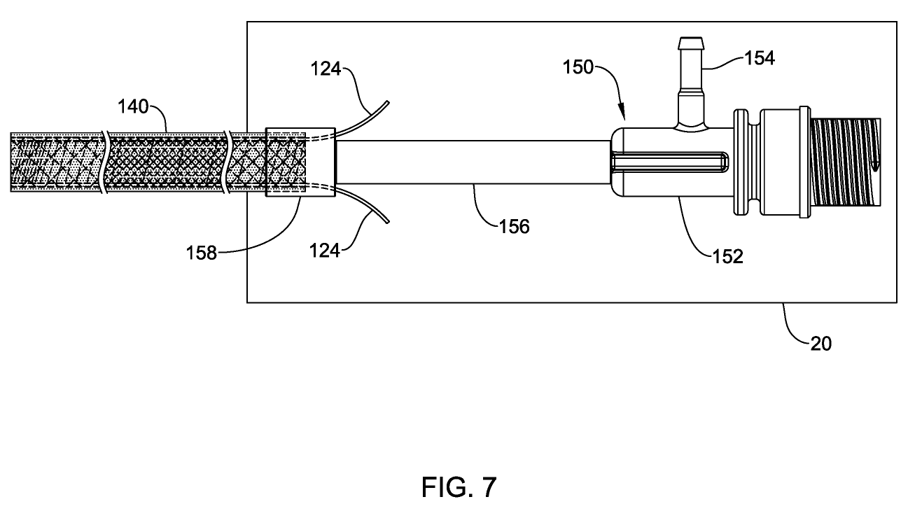

In some embodiments, the method of making the steerable sheath assembly may comprise overmolding a proximal hub structure 150 onto a proximal end of the outer tubular sleeve 140 and/or the portion of the core wire 124 extending proximally out of each tubular member of the at least one tubular member 120, as seen in FIG. 7. In some embodiments, the method of making the steerable sheath assembly may comprise positioning a proximal end of the steerable sheath assembly into a mold 20 and subsequently overmolding the proximal hub structure 150 onto the proximal end of the outer tubular sleeve 140 and/or the portion of the core wire 124 extending proximally out of each tubular member of the at least one tubular member 120. In some embodiments, the method of making the steerable sheath assembly may comprise securing the core wire 124 extending proximal of each tubular member of the at least one tubular member 120 within the mold 20 to orient the core wire 124 extending proximal of each tubular member of the at least one tubular member 120 at a desired angle relative to a central longitudinal axis of the inner tubular sleeve 100 and/or the steerable sheath assembly. In some embodiments, the method of making the steerable sheath assembly may comprise securing the core wire 124 extending proximal of each tubular member of the at least one tubular member 120 within the mold 20 to orient the core wire 124 extending proximal of each tubular member of the at least one tubular member 120 at a desired radius of curvature.

In some embodiments, the method of making the steerable sheath assembly may comprise removing the mandrel or the core 10 from the central lumen of the inner tubular sleeve 100 prior to overmolding the proximal hub structure 150 onto the proximal end of the outer tubular sleeve 140. In some embodiments, the method of making the steerable sheath assembly may comprise removing the mandrel or the core 10 from the central lumen of the inner tubular sleeve 100 after overmolding the proximal hub structure 150 onto the proximal end of the outer tubular sleeve 140.

In some embodiments, the proximal hub structure 150 may comprise a proximal portion 152, a distal portion 158, and a tubular medial portion 156. In some embodiments, the proximal portion 152 may comprise a fluid port 154 spaced apart from the distal portion 158 of the proximal hub structure 150. In some embodiments, the tubular medial portion 156 may extend from the proximal portion 152 of the proximal hub structure 150 to the distal portion 158 of the proximal hub structure 150. In some embodiments, the distal portion 158 may have an outer diameter greater than an outer diameter of the tubular medial portion 156. In at least some embodiments, the proximal portion 152, the tubular medial portion 156, and the distal portion 158 may be monolithically formed with each other as a single structure.

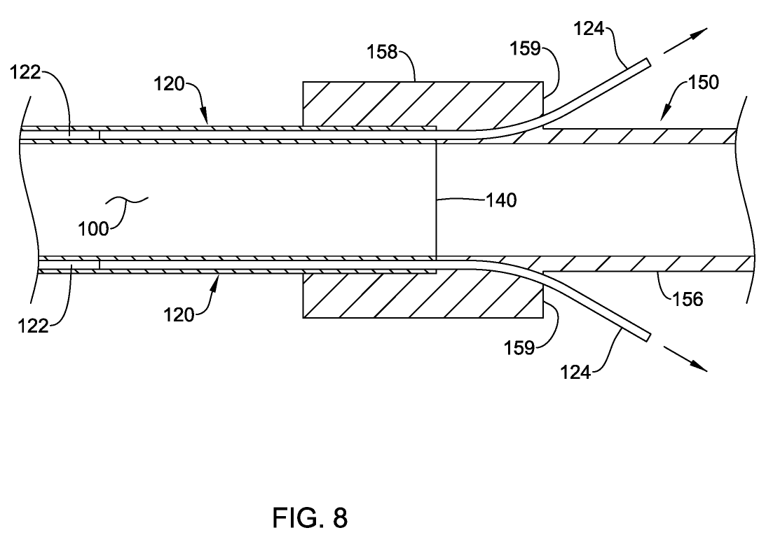

In some embodiments, after overmolding the proximal hub structure 150 onto the proximal end of the outer tubular sleeve 140, the core wire 124 of each tubular member of the at least one tubular member 120 may extend through the distal portion 158 of the proximal hub structure 150 to an exterior of the proximal hub structure 150 at a location distal of a proximal end of the proximal hub structure 150, as seen in FIG. 8. To improve clarity, some elements of the steerable sheath assembly are not shown or are shown schematically. In some embodiments, after overmolding the proximal hub structure 150 onto the proximal end of the outer tubular sleeve 140, the core wire 124 of each tubular member of the at least one tubular member 120 may extend through the distal portion 158 of the proximal hub structure 150 to the exterior of the proximal hub structure 150 at a location distal of a proximal portion 152 (e.g., FIG. 7) of the proximal hub structure 150.

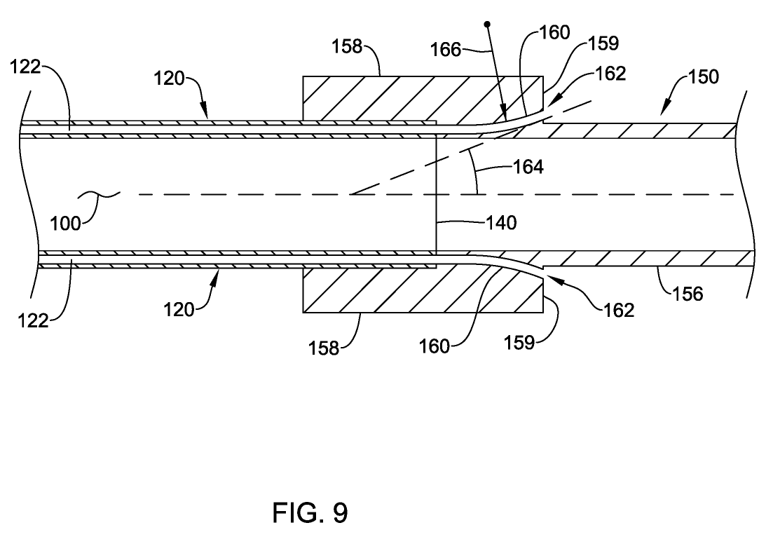

In some embodiments, the method of making the steerable sheath assembly may comprise removing the core wire 124 of each tubular member of the at least one tubular member 120 from the distal portion 158 of the proximal hub structure 150 thereby defining a separate lumen 160 extending from each tubular member of the at least one tubular member 120 through the distal portion 158 of the proximal hub structure 150 to the exterior of the proximal hub structure 150, as seen in FIGS. 8-9. In some embodiments, removing the core wire 124 of each tubular member of the at least one tubular member 120 from the distal portion 158 of the proximal hub structure 150 may comprise applying tension to and/or pulling on a proximal end of the core wire 124 of each tubular member of the at least one tubular member 120.

Accordingly, after removing the core wire 124 of each tubular member of the at least one tubular member 120, the proximal hub structure 150 may comprise a separate lumen 160 extending from each tubular member of the at least one tubular member 120 through the distal portion 158 of the proximal hub structure 150 to the exterior of the proximal hub structure 150 at a location distal of the proximal end of the proximal hub structure and/or at a location distal of the proximal portion 152 of the proximal hub structure 150.

In some embodiments, each separate lumen 160 may terminate at and/or open to the exterior of the proximal hub structure 150 in a proximal face 159 of the distal portion 158 of the proximal hub structure 150. For example, a proximal end 162 of each separate lumen 160 may be disposed at and/or within the proximal face 159 of the distal portion 158 of the proximal hub structure 150.

In some embodiments, each separate lumen 160 extending through the distal portion 158 of the proximal hub structure 150, at the proximal end 162 thereof, may form an angle 164 relative to a central longitudinal axis of the proximal hub structure 150 and/or the central longitudinal axis of the inner tubular sleeve 100 (e.g., in some embodiments, the axes may be coincident and/or coaxial) of between about 0 degrees and about 60 degrees, as seen in FIG. 9. To improve clarity, some elements of the steerable sheath assembly are not shown or are shown schematically. In some embodiments, the angle 164 relative to the central longitudinal axis of the proximal hub structure 150 and/or the central longitudinal axis of the inner tubular sleeve 100 may be between about 0 degrees and about 45 degrees. In some embodiments, the angle 164 relative to the central longitudinal axis of the proximal hub structure 150 and/or the central longitudinal axis of the inner tubular sleeve 100 may be between about 0 degrees and about 35 degrees. In some embodiments, the angle 164 relative to the central longitudinal axis of the proximal hub structure 150 and/or the central longitudinal axis of the inner tubular sleeve 100 may be between about 0 degrees and about 25 degrees. In some embodiments, the angle 164 relative to the central longitudinal axis of the proximal hub structure 150 and/or the central longitudinal axis of the inner tubular sleeve 100 may be between about 0 degrees and about 20 degrees. In some embodiments, the angle 164 relative to the central longitudinal axis of the proximal hub structure 150 and/or the central longitudinal axis of the inner tubular sleeve 100 may be between about 0 degrees and about 15 degrees. In some embodiments, the angle 164 relative to the central longitudinal axis of the proximal hub structure 150 and/or the central longitudinal axis of the inner tubular sleeve 100 may be about 5 degrees, about 10 degrees, about 15 degrees, about 20 degrees, about 25 degrees, about 30 degrees, etc.

In some embodiments, each separate lumen 160 extending through the distal portion 158 of the proximal hub structure 150 may curve within the distal portion 158 of the proximal hub structure 150 along a radius 166 of between about 0.050 inches (1.27 millimeters (mm)) and about 0.500 inches (12.7 mm), as seen in FIG. 9. In some embodiments, the radius 166 may be between about 0.075 inches (1.91 mm) and about 0.400 inches (10.16 mm). In some embodiments, the radius 166 may be between about 0.100 inches (2.54 mm) and about 0.300 inches (7.62 mm). In some embodiments, the radius 166 may be between about 0.125 inches (3.18 mm) and about 0.250 inches (6.35 mm). In some embodiments, the radius 166 may be between about 0.150 inches (3.81) and about 0.200 inches (5.08 mm). In some embodiments, the radius 166 may be about 0.075 inches (1.91 mm), about 0.100 inches (2.54 mm), about 0.150 inches (3.81 mm), about 0.200 inches (5.08 mm), about 0.250 inches (6.35 mm), about 0.300 inches (7.62 mm), about 0.400 inches (10.16 mm), etc.

In some embodiments, the radius 166 may extend along an entire length of the separate lumen 160 (e.g., the separate lumen 160 may form a continuous curve or arc). In some embodiments, the radius 166 may extend along only a portion of the separate lumen 160. In one example, the separate lumen 160 may comprise a substantially straight proximal section adjacent the proximal end 162 of the separate lumen 160 with the radius 166 disposed distally of the substantially straight proximal section. In another example, the separate lumen 160 may comprise a substantially straight distal section adjacent the distal end of the separate lumen 160 with the radius 166 disposed proximally of the substantially straight distal section. In yet another example, the separate lumen 160 may comprise a substantially straight proximal section adjacent the proximal end 162 of the separate lumen 160 and a substantially straight distal section adjacent the distal end of the separate lumen 160 with the radius 166 disposed therebetween.

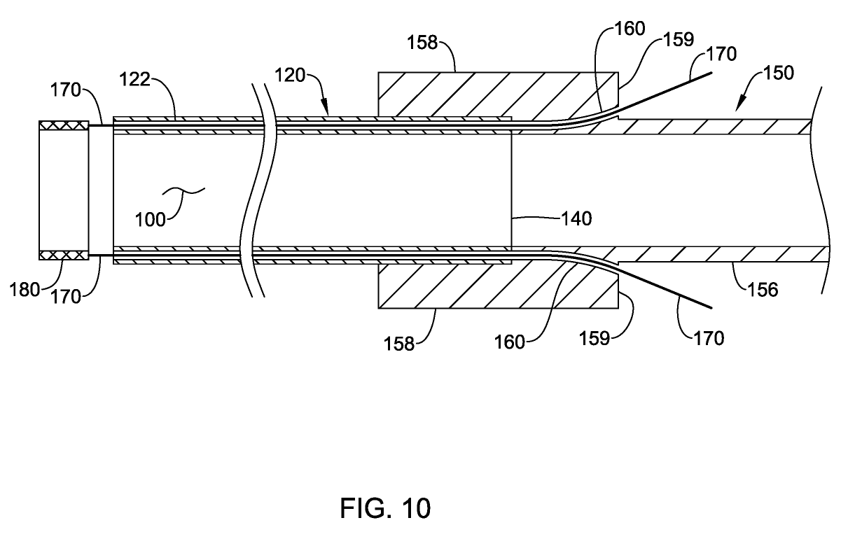

In some embodiments, the method of making the steerable sheath assembly may comprise, after removing the core wire 124 of each tubular member of the at least one tubular member 120, positioning a steering wire 170 within each tubular member of the at least one tubular member 120 such that each steering wire 170 extends distal from its respective tubular member of the at least one tubular member 120, through its respective tubular member of the at least one tubular member 120, and exits to the exterior of the proximal hub structure 150 through its respective separate lumen 160, as seen in FIG. 10. To improve clarity, some elements of the steerable sheath assembly are not shown or are shown schematically. Each steering wire 170 may be slidably disposed within its respective tubular member of the at least one tubular member 120. For example, there may be sufficient clearance around each steering wire 170 within its respective tubular member of the at least one tubular member 120 to permit each steering wire 170 to axially translate relative to its respective tubular member of the at least one tubular member 120.

Accordingly, the steerable sheath assembly may comprise a steering wire 170 disposed within each tubular member of the at least one tubular member 120 such that each steering wire 170 extends from the exterior of the proximal hub structure 150 to distal of the at least one tubular member 120. The steerable sheath assembly may comprise a steering ring 180 disposed proximate a distal end of the reinforcing braid 130 and/or a distal end of the outer tubular sleeve 140. Each steering wire 170 may be fixedly attached to the steering ring 180. In some embodiments, the steering wire 170 and/or the steering ring 180 may be formed from a metallic material. In one non-limiting example, the steering wire 170 and/or the steering ring 180 may be formed from stainless steel. Other configurations and/or materials are also contemplated. Some suitable but non-limiting examples of materials that may be used for the steering wire 170 and/or the steering ring 180, including polymeric materials, metallic materials, composites, and the like, are described below.

In some embodiments, positioning a steering wire 170 within each tubular member of the at least one tubular member 120 may comprise inserting a steering wire 170 within the distal end of each tubular member of the at least one tubular member 120 and advancing each steering wire 170 proximally within its respective tubular member of the at least one tubular member 120 to the exterior of the proximal hub structure 150.

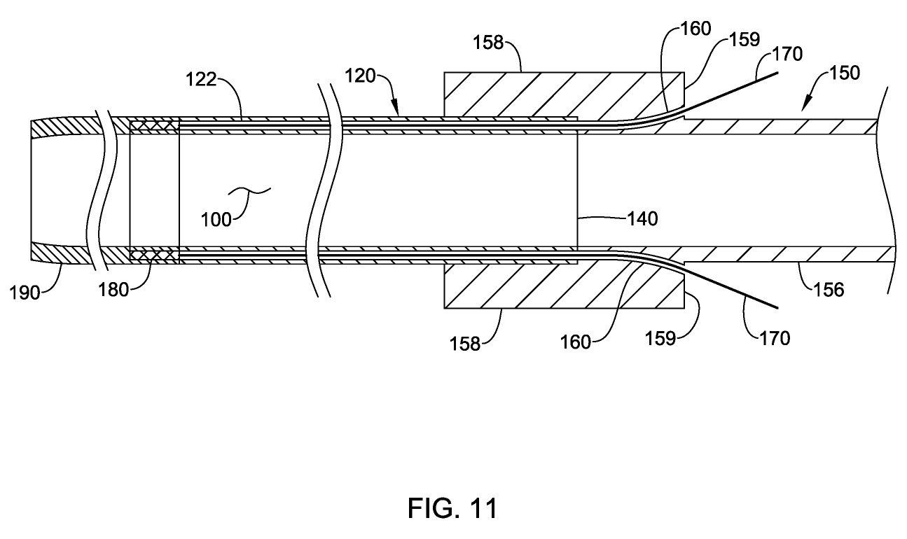

In some embodiments, the method of making the steerable sheath assembly may comprise attaching a steering ring 180 to the inner tubular sleeve 100 and/or the outer tubular sleeve 140 proximate a distal end of the reinforcing braid 130, proximate a distal end of the inner tubular sleeve 100, and/or proximate a distal end of the outer tubular sleeve 140, as seen in FIG. 11. To improve clarity, some elements of the steerable sheath assembly are not shown or are shown schematically. In some embodiments, the steering ring 180 may be attached directly to the inner tubular sleeve 100 and/or the outer tubular sleeve 140. In some embodiments, the steering ring 180 may be disposed radially outward of and/or on an outer surface of the inner tubular sleeve 100. In some embodiments, the steering ring 180 may be disposed distal of the outer tubular sleeve 140 and/or the reinforcing braid 130.

In some embodiments, the method of making the steerable sheath assembly may comprise forming a distal tip portion 190 and/or attaching a distal tip portion 190 thereto, as seen in FIG. 11. To improve clarity, some elements of the steerable sheath assembly are not shown or are shown schematically. In some embodiments, the steering ring 180 may be disposed on and/or within the distal tip portion 190. In some embodiments, the steering ring 180 may be embedded within the distal tip portion 190. In some embodiments, the distal tip portion 190 may be formed from a polymeric material. In some embodiments, the distal tip portion 190 may comprise polyether block amide (e.g., Pebax®). Other configurations and/or polymeric materials are also contemplated, and some non-limiting examples are described below.

In some embodiments, the distal tip portion 190 may be fixedly attached to the inner tubular sleeve 100 and/or the outer tubular sleeve 140. In some embodiments, the distal tip portion 190 may be adhesively bonded to the inner tubular sleeve 100 and/or the outer tubular sleeve 140. In some embodiments, the distal tip portion 190 may be welded to the inner tubular sleeve 100 and/or the outer tubular sleeve 140. In some embodiments, the distal tip portion 190 may be melt-bonded to the inner tubular sleeve 100 and/or the outer tubular sleeve 140. In some embodiments, the distal tip portion 190 may be reflowed with the inner tubular sleeve 100 and/or the outer tubular sleeve 140. Other configurations are also contemplated.

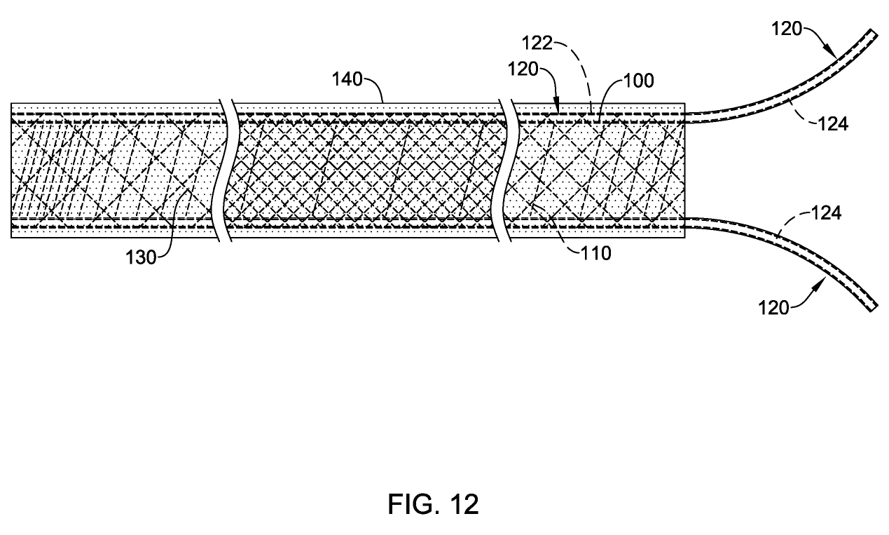

FIGS. 12-14 illustrate selected aspects of a steerable sheath assembly and a method of making the steerable sheath assembly. It shall be noted that at least some aspects overlap with the steerable sheath assembly and method of FIGS. 1-11. As such, FIGS. 12-14 illustrate additional and/or alternative aspects thereof. For example, aspects of the steerable sheath assembly and the method of making the steerable sheath assembly generally shown in and/or discussed previously also apply and/or are similar. To improve clarity, some elements of the steerable sheath assembly are not shown or are shown schematically.

In some embodiments, disposing at least one tubular member 120 alongside the inner tubular sleeve 100 and/or the helical coil 110 may comprise disposing the at least one tubular member 120 alongside the inner tubular sleeve 100 and/or the helical coil 110 such that the at least one tubular member 120 extends proximal of a proximal end of the inner tubular sleeve 100 and/or the helical coil 110. In some embodiments, each tubular member of the at least one tubular member 120 may comprise the core wire 124 disposed therein when disposing the at least one tubular member 120 alongside the inner tubular sleeve 100 and/or the helical coil 110. In some embodiments, disposing the reinforcing braid 130 over the inner tubular sleeve 100 and the at least one tubular member 120 may comprise disposing the reinforcing braid 130 over the inner tubular sleeve 100 and the at least one tubular member 120 such that the at least one tubular member 120 extends proximal of the reinforcing braid 130. In some embodiments, disposing the outer tubular sleeve 140 over the reinforcing braid 130 may comprise disposing the outer tubular sleeve 140 over the reinforcing braid 130 such that the at least one tubular member 120 extends proximal of the outer tubular sleeve 140.

Accordingly, in some embodiments, the at least one tubular member 120 may extend proximal of a proximal end of the outer tubular sleeve 140, a proximal end of the reinforcing braid 130, a proximal end of the helical coil 110, and/or a proximal end of the inner tubular sleeve 100, as seen in FIG. 12. In some embodiments, the steerable sheath assembly may be constructed such that all elements of the steerable sheath assembly, except for the at least one tubular member 120 (and the core wire 124 disposed therein), terminate at a single longitudinal position at the proximal end of the outer tubular sleeve 140. In some embodiments, the steerable sheath assembly may be trimmed to length such that all elements of the steerable sheath assembly, except for the at least one tubular member 120 (and the core wire 124 disposed therein), terminate at a single longitudinal position at the proximal end of the outer tubular sleeve 140. Other configurations are also contemplated.

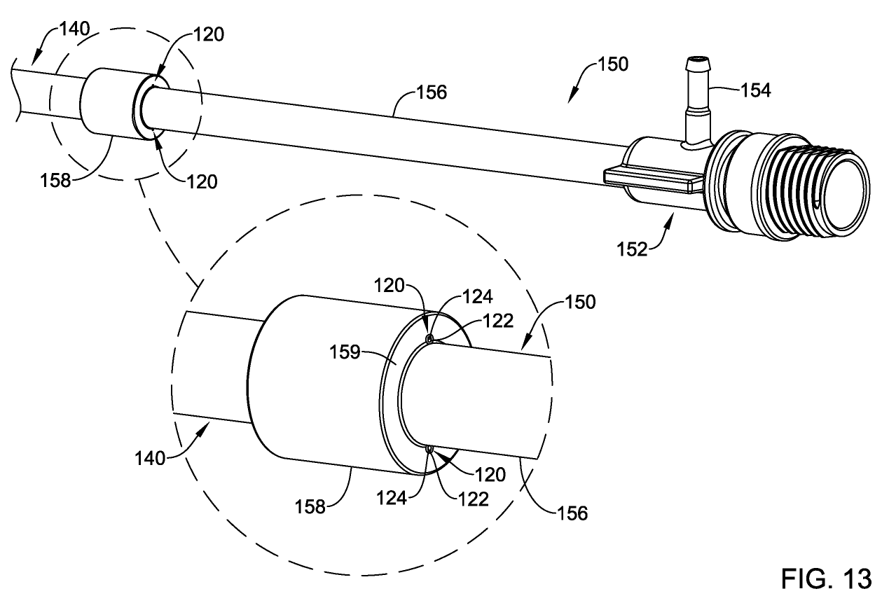

In some embodiments, the method of making the steerable sheath assembly may comprise overmolding the proximal hub structure 150 onto the proximal end of the outer tubular sleeve 140 and a proximal portion of each tubular member of the at least one tubular member 120, as seen in FIG. 13. In some embodiments, the method of making the steerable sheath assembly may comprise positioning the proximal end of the steerable sheath assembly into the mold 20 (e.g., similar to FIG. 7 above) and subsequently overmolding the proximal hub structure 150 onto the proximal end of the outer tubular sleeve 140 and the proximal portion of each tubular member of the at least one tubular member 120. In some embodiments, the method of making the steerable sheath assembly may comprise securing the proximal portion of each tubular member of the at least one tubular member 120 extending proximal of the outer tubular sleeve 140 within the mold 20 (e.g., similar to FIG. 7 above) to orient the proximal portion of each tubular member of the at least one tubular member 120 extending proximal of the outer tubular sleeve 140 at a desired angle relative to a central longitudinal axis of the inner tubular sleeve 100 and/or the steerable sheath assembly, similar to FIG. 9 above. In some embodiments, the method of making the steerable sheath assembly may comprise securing the proximal portion of each tubular member of the at least one tubular member 120 extending proximal of the outer tubular sleeve 140 within the mold 20 to orient the proximal portion of each tubular member of the at least one tubular member 120 extending proximal of the outer tubular sleeve 140 at a desired radius of curvature, similar to FIG. 9 above.

In some embodiments, the method of making the steerable sheath assembly may comprise overmolding the proximal hub structure 150 onto the proximal end of the outer tubular sleeve 140 and the proximal portion of each tubular member of the at least one tubular member 120 with the core wire 124 disposed within each tubular member of the at least one tubular member 120. In some embodiments, the method of making the steerable sheath assembly may comprise removing the core wire 124 from each tubular member of the at least one tubular member 120 prior to overmolding the proximal hub structure 150 onto the proximal end of the outer tubular sleeve 140 and the proximal portion of each tubular member of the at least one tubular member 120.

In some embodiments, the method of making the steerable sheath assembly may comprise removing the mandrel or the core 10 from the central lumen of the inner tubular sleeve 100 prior to overmolding the proximal hub structure 150 onto the proximal end of the outer tubular sleeve 140 and the proximal portion of each tubular member of the at least one tubular member 120. In some embodiments, the method of making the steerable sheath assembly may comprise removing the mandrel or the core 10 from the central lumen of the inner tubular sleeve 100 after overmolding the proximal hub structure 150 onto the proximal end of the outer tubular sleeve 140 and the proximal portion of each tubular member of the at least one tubular member 120.

In some embodiments, after overmolding the proximal hub structure 150 onto the proximal end of the outer tubular sleeve 140 and the proximal portion of each tubular member of the at least one tubular member 120, the proximal portion of each tubular member of the at least one tubular member 120 may extend through the distal portion 158 of the proximal hub structure 150 to the proximal face 159 of the distal portion 158 of the proximal hub structure and/or to the exterior of the proximal hub structure 150 at a location distal of a proximal end of the proximal hub structure 150, as seen in FIG. 13. In some embodiments, after overmolding the proximal hub structure 150 onto the proximal end of the outer tubular sleeve 140 and the proximal portion of each tubular member of the at least one tubular member 120, the proximal portion of each tubular member of the at least one tubular member 120 may extend through the distal portion 158 of the proximal hub structure 150 to the exterior of the proximal hub structure 150 at a location distal of a proximal portion 152 of the proximal hub structure 150.

In some embodiments, the steering wire lumen 122 of each tubular member of the at least one tubular member 120 may exit and/or open to the exterior of the proximal hub structure 150 at a location distal of the proximal end of the proximal hub structure and/or at a location distal of the proximal portion 152 of the proximal hub structure 150. For example, the steering wire lumen 122 may form the separate lumen 160 discussed above and/or may be used interchangeably therewith. In some embodiments, each steering wire lumen 122 may terminate at and/or open to the exterior of the proximal hub structure 150 in the proximal face 159 of the distal portion 158 of the proximal hub structure 150. For example, a proximal end of each steering wire lumen 122 may be disposed at and/or within the proximal face 159 of the distal portion 158 of the proximal hub structure 150, as seen in FIG. 13.

In some embodiments, each steering wire lumen 122 extending through the distal portion 158 of the proximal hub structure 150, at the proximal end thereof, may form an angle relative to a central longitudinal axis of the proximal hub structure 150 and/or the central longitudinal axis of the inner tubular sleeve 100 (e.g., in some embodiments, the axes may be coincident and/or coaxial). In some embodiments, each steering wire lumen 122 extending through the distal portion 158 of the proximal hub structure 150 may curve within the distal portion 158 of the proximal hub structure 150 along a radius. In at least some embodiments, the angle 164 and/or the radius 166 discussed above with respect to FIG. 9, and/or characteristics, values, sizes, etc. thereof, may also apply to the configuration shown in FIGS. 12-14.

In some embodiments, the method of making the steerable sheath assembly may comprise positioning a steering wire 170 within the steering wire lumen 122 of each tubular member of the at least one tubular member 120 after removal of the core wire 124 such that each steering wire 170 extends from proximal of its respective tubular member of the at least one tubular member 120 to distal of its respective tubular member of the at least one tubular member 120, similar to FIG. 10 above. Each steering wire 170 may be slidably disposed within the steering wire lumen 122 of its respective tubular member of the at least one tubular member 120. For example, there may be sufficient clearance around each steering wire 170 within the steering wire lumen 122 of its respective tubular member of the at least one tubular member 120 to permit each steering wire 170 to axially translate relative to its respective tubular member of the at least one tubular member 120.

Accordingly, the steerable sheath assembly may comprise a steering wire 170 disposed within the steering wire lumen 122 of each tubular member of the at least one tubular member 120 such that each steering wire 170 extends from the exterior of the proximal hub structure 150 to distal of the at least one tubular member 120. The steerable sheath assembly may comprise a steering ring 180 disposed proximate the distal end of the inner tubular sleeve 100, the distal end of the reinforcing braid 130, and/or the distal end of the outer tubular sleeve 140. Each steering wire 170 may be fixedly attached to the steering ring 180.

In some embodiments, positioning the steering wire 170 within the steering wire lumen 122 of each tubular member of the at least one tubular member 120 may comprise inserting the steering wire 170 within the distal end of the steering wire lumen 122 of each tubular member of the at least one tubular member 120 and advancing each steering wire 170 proximally within the steering wire lumen 122 of its respective tubular member of the at least one tubular member 120 to the exterior of the proximal hub structure 150.

In some embodiments, the method of making the steerable sheath assembly may comprise attaching the steering ring 180 to the inner tubular sleeve 100 and/or the outer tubular sleeve 140 proximate the distal end of the reinforcing braid 130, proximate the distal end of the inner tubular sleeve 100, and/or proximate the distal end of the outer tubular sleeve 140, similar to FIG. 11 above. In some embodiments, the steering ring 180 may be attached directly to the inner tubular sleeve 100 and/or the outer tubular sleeve 140. In some embodiments, the steering ring 180 may be disposed radially outward of and/or on an outer surface of the inner tubular sleeve 100. In some embodiments, the steering ring 180 may be disposed distal of the outer tubular sleeve 140 and/or the reinforcing braid 130.

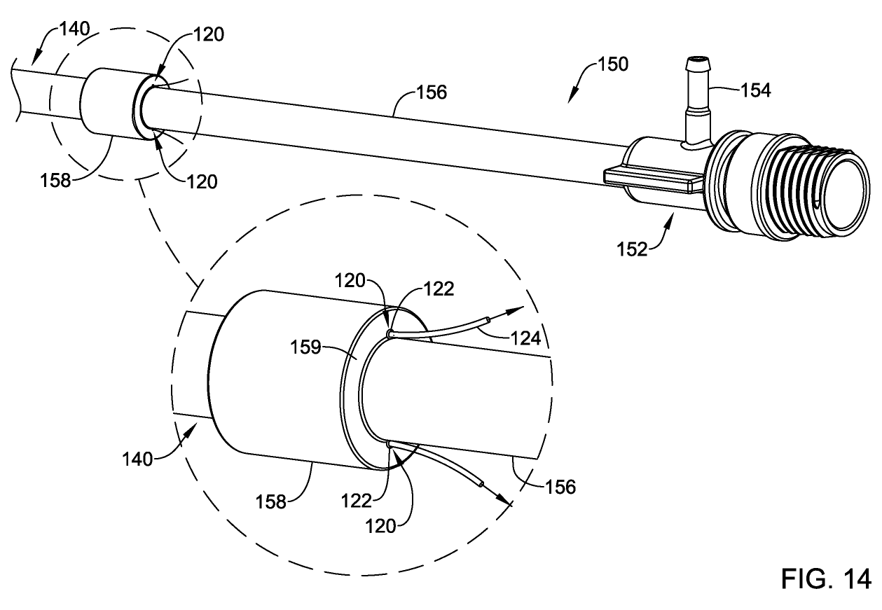

In some embodiments, the method of making the steerable sheath assembly may comprise, after overmolding the proximal hub structure 150 and before positioning the steering wire 170 within each tubular member of the at least one tubular member 120, removing the core wire 124 from the steering wire lumen 122 of each tubular member of the at least one tubular member 120, as seen in FIG. 14. In some embodiments, having the at least one tubular member 120 disposed within and/or extending through the proximal hub structure 150 may permit any coating (e.g., lubricious, etc.) disposed on the inner surface of the steering wire lumen 122 to be present within the proximal hub structure 150 without additional processing.

In some embodiments, the method of making the steerable sheath assembly may comprise forming a distal tip portion 190 and/or attaching a distal tip portion 190 thereto, similar to FIG. 11 above. In some embodiments, the steering ring 180 may be disposed on and/or within the distal tip portion 190. In some embodiments, the steering ring 180 may be embedded within the distal tip portion 190. In some embodiments, the distal tip portion 190 may be formed from a polymeric material. In some embodiments, the distal tip portion 190 may comprise polyether block amide (e.g., Pebax®). Other configurations and/or polymeric materials are also contemplated, and some non-limiting examples are described below.

In some embodiments, the distal tip portion 190 may be fixedly attached to the inner tubular sleeve 100 and/or the outer tubular sleeve 140. In some embodiments, the distal tip portion 190 may be adhesively bonded to the inner tubular sleeve 100 and/or the outer tubular sleeve 140. In some embodiments, the distal tip portion 190 may be welded to the inner tubular sleeve 100 and/or the outer tubular sleeve 140. In some embodiments, the distal tip portion 190 may be melt-bonded to the inner tubular sleeve 100 and/or the outer tubular sleeve 140. In some embodiments, the distal tip portion 190 may be reflowed with the inner tubular sleeve 100 and/or the outer tubular sleeve 140. Other configurations are also contemplated.

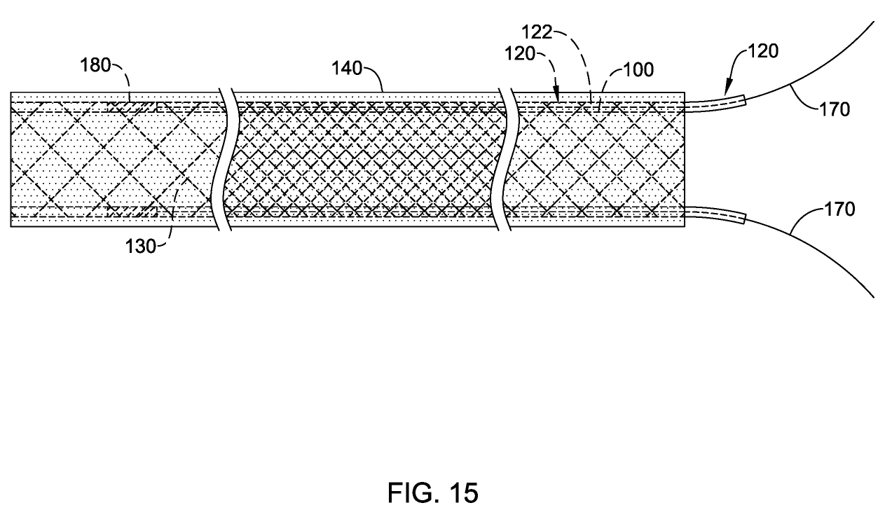

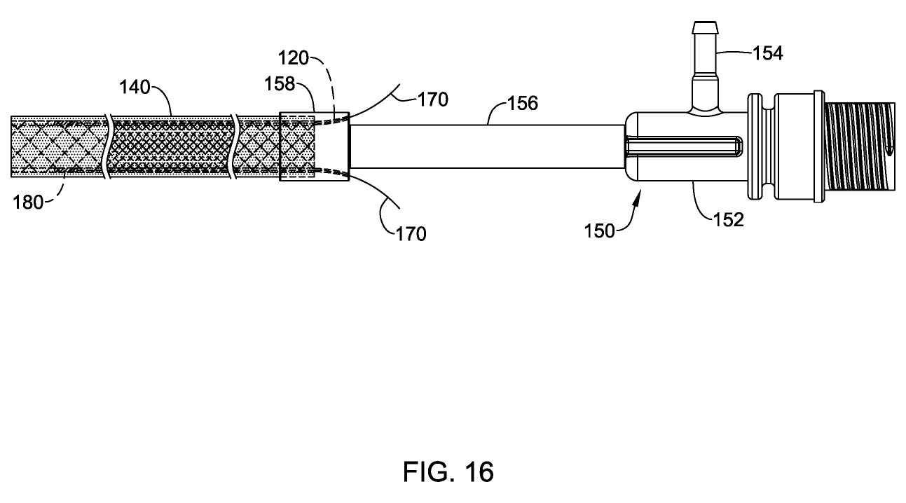

FIGS. 15-16 illustrate selected aspects of a steerable sheath assembly and a method of making the steerable sheath assembly. It shall be noted that at least some aspects overlap with the steerable sheath assembly and method of FIGS. 1-11 and/or FIGS. 12-14. As such, FIGS. 15-16 illustrate additional and/or alternative aspects thereof. For example, aspects of the steerable sheath assembly and the method of making the steerable sheath assembly generally shown in and/or discussed previously also apply and/or are similar. To improve clarity, some elements of the steerable sheath assembly are not shown or are shown schematically.

In some embodiments, the steerable sheath assembly may be constructed slightly differently and/or using steps performed in a different order. In some embodiments, the method of making the steerable sheath assembly may comprise, before disposing the reinforcing braid 130 over the inner tubular sleeve 100 and the at least one tubular member 120, positioning the steering wire 170 within each tubular member of the at least one tubular member 120 such that each steering wire 170 extends from proximal of its respective tubular member of the at least one tubular member 120 to distal of its respective tubular member of the at least one tubular member 120. In some embodiments, the method of making the steerable sheath assembly may comprise positioning the steering ring 180 over the inner tubular sleeve 100 and/or the helical coil 110 distal of the at least one tubular member 120. To facilitate this, in some embodiments, the core wire 124 may be removed from each tubular member of the at least one tubular member 120 prior to positioning the steering wire 170 within each tubular member of the at least one tubular member 120. Alternatively, in some embodiments, the at least one tubular member 120 may be positioned alongside the inner tubular sleeve 100 and/or the helical coil 110 without any core wire disposed therein.

In some embodiments, disposing the reinforcing braid 130 over the inner tubular sleeve 100, the helical coil 110, and the at least one tubular member 120 may comprise disposing the reinforcing braid 130 over the steering ring 180, as seen in FIG. 15 for example. In some embodiments, disposing the reinforcing braid 130 over the inner tubular sleeve 100, the helical coil 110, the at least one tubular member 120, and the steering ring 180 may secure the steering ring 180 to the inner tubular sleeve 100 and/or may secure the steering ring 180 in place along the length of the inner tubular sleeve 100. In some embodiments, disposing the outer tubular sleeve 140 over the inner tubular sleeve 100, the at least one tubular member 120, the steering ring 180, and/or the reinforcing braid 130 may secure the steering ring 180 to the inner tubular sleeve 100 and/or may secure the steering ring 180 in place along the length of the inner tubular sleeve 100. Other configurations are also contemplated.

In some embodiments, the method of making the steerable sheath assembly may comprise overmolding the proximal hub structure 150 onto the proximal end of the outer tubular sleeve 140 and the proximal portion of each tubular member of the at least one tubular member 120 with the steering wire 170 disposed within each tubular member of the at least one tubular member 120, as seen in FIG. 16. In some embodiments, the method of making the steerable sheath assembly may comprise inserting a support element (not shown) into the steering wire lumen 122 of each tubular member of the at least one tubular member 120 prior to overmolding the proximal hub structure 150 onto the proximal end of the outer tubular sleeve 140 and the proximal portion of each tubular member of the at least one tubular member 120. In some embodiments, the support element may maintain and/or hold open the steering wire lumen 122 during the overmolding process. In some embodiments, the support element may be advanced over the steering wire 170 into the steering wire lumen 122 of each tubular member of the at least one tubular member 120. In some embodiments, the support element may be advanced alongside the steering wire 170 into the steering wire lumen 122 of each tubular member of the at least one tubular member 120.

In any embodiment and/or configuration discussed herein, each steering wire 170 may be configured to be attached to and/or coupled to a steering mechanism and/or a steering actuator. The steering mechanism and/or the steering actuator may be disposed within and/or may be operably coupled to a handle of medical device disposed at and/or proximal the proximal end of the steerable sheath assembly. In some embodiments, at least a portion of the proximal hub structure 150 may be disposed within the handle. In some embodiments, a majority of the proximal hub structure 150 may be disposed within the handle. Other configurations are also contemplated. Tension may be selectively applied to each steering wire 170 using the steering mechanism and/or the steering actuator to bend and/or deflect a distal portion the steerable sheath assembly.

In some embodiments, the steering wire lumen 122 of each tubular member of the at least one tubular member 120 may have a circular cross-sectional shape. However, this is merely exemplary and is not intended to be limiting. In some embodiments, the steering wire lumen 122 of each tubular member of the at least one tubular member 120 may have other cross-sectional shapes. For example, in some embodiments, the steering wire lumen 122 of each tubular member of the at least one tubular member 120 may have a rectangular cross-sectional shape, an ovoid cross-sectional shape, a square cross-sectional shape, a polygonal cross-sectional shape, etc. In some embodiments, the steering wire lumen 122 of each tubular member of the at least one tubular member 120 may have a cross-sectional shape that is regular and/or symmetrical. In some embodiments, the steering wire lumen 122 of each tubular member of the at least one tubular member 120 may have a cross-sectional shape that is irregular and/or asymmetrical. Other configurations are also contemplated.

In some embodiments, the steerable sheath assembly may be sized in accordance with its intended use. For example, the steerable sheath assembly can have a length that is in the range of about 50 to about 200 centimeters, about 75 to about 175 centimeters, or about 100 to about 150 centimeters. Other lengths are also contemplated. It is further contemplated that the outer diameter of the steerable sheath assembly and/or the outer tubular sleeve 140 may vary based on the use or application. In some examples, the outer diameter of the steerable sheath assembly and/or the outer tubular sleeve 140 may be about 2 millimeters (mm), about 3 mm (or 9 French), about 3.5 mm, about 4 mm (or 12 French), about 4.5 mm, about 5 mm (or 15 French), about 5.33 mm, about 5.5 mm, about 5.66 mm (or 17 French), about 6 mm, about 6.5 mm, about 7 mm (or 21 French), about 8 mm, or other suitable sizes. In some embodiments, the outer diameter of the steerable sheath assembly and/or the outer tubular sleeve 140 may be a maximum of 5.66 mm (17 French), and is preferably smaller than 5.66 mm (17 French). Other configurations are also contemplated.

In some embodiments, the steerable sheath assembly and/or components thereof may be heat treated to form and/or define the normal configuration or the relaxed configuration. In some embodiments, the steerable sheath assembly may include a preset double curve in the normal configuration or the relaxed configuration. In some embodiments, the method of making the steerable sheath assembly may comprise heat treating the steerable sheath assembly and/or the components thereof to form and/or define the normal configuration or the relaxed configuration. In some embodiments, the method of making the steerable sheath assembly may comprise heat treating the steerable sheath assembly and/or the components thereof before overmolding the proximal hub structure 150 to the outer tubular sleeve 140. In some embodiments, the method of making the steerable sheath assembly may comprise heat treating the steerable sheath assembly and/or the components thereof after fixedly attaching the proximal hub structure 150 to the outer tubular sleeve 140. Other configurations and/or materials are also contemplated.

The materials that can be used for the various components of the assembly (and/or other elements disclosed herein) and the various components thereof disclosed herein may include those commonly associated with medical devices and/or systems. For simplicity purposes, the following discussion refers to the assembly. However, this is not intended to limit the devices and methods described herein, as the discussion may be applied to other elements, members, components, or devices disclosed herein, such as, but not limited to, the inner tubular sleeve, the helical coil, the at least one tubular member, the reinforcing braid, the outer tubular sleeve, the steering wire, the core wire, the steering ring, etc. and/or elements or components thereof.

In some embodiments, the assembly and/or components thereof may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP).

Some examples of suitable metals and metal alloys include stainless steel, such as 304 and/or 316 stainless steel and/or variations thereof; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the assembly and/or components thereof may also be doped with, made of, or otherwise include a radiopaque material. Radiopaque materials are understood to be materials capable of producing a relatively dark image on a fluoroscopy screen or another imaging technique (e.g., ultrasound, etc.) during a medical procedure. This relatively dark image aids the user of the assembly in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the assembly to achieve the same result.

In some embodiments, the assembly and/or components thereof may include a fabric material. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the assembly and/or components thereof may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum or a Ni-Co-Cr-based alloy. The yarns may further include carbon, glass or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the assembly and/or components thereof may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A method of making a steerable sheath assembly, comprising:
positioning an inner tubular sleeve on a mandrel;
disposing at least one tubular member alongside the inner tubular sleeve, each tubular member of the at least one tubular member having a core wire disposed therein;
disposing a reinforcing braid over the inner tubular sleeve and the at least one tubular member;
disposing an outer tubular sleeve over the reinforcing braid;
proximally withdrawing the core wire from each tubular member of the at least one tubular member only partially from its respective tubular member; and
overmolding a proximal hub structure onto a proximal end of the outer tubular sleeve;
wherein the core wire of each tubular member of the at least one tubular member extends through a distal portion of the proximal hub structure to an exterior of the proximal hub structure at a location distal of a proximal end of the proximal hub structure.

2. The method of claim 1, further comprising:
removing the core wire of each tubular member of the at least one tubular member from the distal portion of the proximal hub structure thereby defining a separate lumen extending from each tubular member of the at least one tubular member through the distal portion of the proximal hub structure to the exterior of the proximal hub structure.

3. The method of claim 2, further comprising:
positioning a steering wire within each tubular member of the at least one tubular member such that each steering wire extends distal from its respective tubular member of the at least one tubular member, through its respective tubular member of the at least one tubular member, and exits to the exterior of the proximal hub structure through its respective separate lumen; and
attaching a steering ring to the outer tubular sleeve proximate a distal end of the reinforcing braid;
wherein each steering wire is fixedly attached to the steering ring.

4. The method of claim 2, wherein each separate lumen extending through the distal portion of the proximal hub structure, at a proximal end thereof, forms an angle relative to a central longitudinal axis of the proximal hub structure of between about 0 degrees and about 35 degrees.

5. The method of claim 2, wherein each separate lumen extending through the distal portion of the proximal hub structure curves within the distal portion of the proximal hub structure along a radius of between about 0.075 inches (1.91 mm) and about 0.500 inches (12.7 mm).

6. The method of claim 1, wherein each tubular member of the at least one tubular member does not extend proximal of a proximal end of the outer tubular sleeve.

7. The method of claim 1, further comprising:
disposing a helical coil around the inner tubular sleeve prior to disposing the at least one tubular member alongside the inner tubular sleeve such that the helical coil is disposed between the at least one tubular member and the inner tubular sleeve.

8. A steerable sheath assembly, comprising:
an inner tubular sleeve;
at least one tubular member positioned alongside the inner tubular sleeve;
a reinforcing braid disposed over the inner tubular sleeve and the at least one tubular member;
an outer tubular sleeve disposed over the reinforcing braid; and
a proximal hub structure overmolded onto the outer tubular sleeve;
wherein the proximal hub structure comprises a separate lumen extending from each tubular member of the at least one tubular member through a distal portion of the proximal hub structure to an exterior of the proximal hub structure at a location distal of a proximal end of the proximal hub structure.

9. The steerable sheath assembly of claim 8, further comprising a helical coil between the inner tubular sleeve and the at least one tubular member.

10. The steerable sheath assembly of claim 8, further comprising:
a steering wire disposed within each tubular member of the at least one tubular member such that each steering wire extends from the exterior of the proximal hub structure to distal of the at least one tubular member; and
a steering ring disposed proximate a distal end of the reinforcing braid;
wherein each steering wire is fixedly attached to the steering ring.

11. The steerable sheath assembly of claim 8, wherein each separate lumen extending through the distal portion of the proximal hub structure, at a proximal end thereof, forms an angle relative to a central longitudinal axis of the proximal hub structure of between about 0 degrees and about 35 degrees.

12. The steerable sheath assembly of claim 8, wherein each separate lumen extending through the distal portion of the proximal hub structure curves within the distal portion of the proximal hub structure along a radius of between about 0.075 inches (1.91 mm) and about 0.500 inches (12.7 mm).

13. The steerable sheath assembly of claim 8, wherein the proximal hub structure comprises a proximal portion including a fluid port spaced apart from the distal portion, and a tubular medial portion extending from the proximal portion of the proximal hub structure to the distal portion of the proximal hub structure.

14. A method of making a steerable sheath assembly, comprising:
positioning an inner tubular sleeve on a mandrel;
disposing at least one tubular member alongside the inner tubular sleeve, each tubular member of the at least one tubular member having a core wire disposed therein;
disposing a reinforcing braid over the inner tubular sleeve and the at least one tubular member;
disposing an outer tubular sleeve over the reinforcing braid; and
overmolding a proximal hub structure onto a proximal end of the outer tubular sleeve and a proximal portion of each tubular member of the at least one tubular member;
wherein the proximal portion of each tubular member of the at least one tubular member extends through a distal portion of the proximal hub structure to an exterior of the proximal hub structure at a location distal of a proximal end of the proximal hub structure.

15. The method of claim 14, further comprising, before disposing the reinforcing braid over the inner tubular sleeve and the at least one tubular member:
positioning a steering wire within each tubular member of the at least one tubular member such that each steering wire extends from proximal of its respective tubular member to distal of its respective tubular member; and
positioning a steering ring over inner tubular sleeve distal of the at least one tubular member;
wherein each steering wire is fixedly attached to the steering ring.

16. The method of claim 14, further comprising:
positioning a steering wire within each tubular member of the at least one tubular member such that each steering wire extends from proximal of its respective tubular member to distal of its respective tubular member; and
attaching a steering ring to the outer tubular sleeve proximate a distal end of the reinforcing braid;
wherein each steering wire is fixedly attached to the steering ring.

17. The method of claim 16, wherein each tubular member of the at least one tubular member comprises a core wire disposed therein when disposing the at least one tubular member alongside the inner tubular sleeve.

18. The method of claim 17, further comprising:
after overmolding the proximal hub structure and before positioning the steering wire within each tubular member of the at least one tubular member, removing the core wire from each tubular member of the at least one tubular member.

19. The method of claim 14, wherein the proximal portion of each tubular member of the at least one tubular member at a proximal end thereof forms an angle relative to a central longitudinal axis of the proximal hub structure of between about 0 degrees and about 35 degrees.

20. The method of claim 14, wherein the proximal portion of each tubular member of the at least one tubular member curves within the distal portion of the proximal hub structure along a radius of between about 0.075 inches (1.91 mm) and about 0.500 inches (12.7 mm).
